(19) United States
(12) Patent Application Publication
(10) Pub. No.: US 2006/0235005 A1
(43) Pub. Date: Oct. 19, 2006

(54) Use of phosphodiesterase 5 (PDE5) inhibitors in the treatment of schizophrenia

(75) Inventor: Donald C. Goff, Marblehead, MA (US)

Correspondence Address: GEORGE MACK RIDDLE, 30 GREENFIELD DRIVE, MORAGA, CA, 94556, US

(73) Assignee: Oak Labs, Corp.

(21) Appl. No.: 11/199,303

(22) Filed: Aug. 8, 2005

Related U.S. Application Data
(60) Provisional application No. 60/671,198, filed on Apr. 14, 2005.

(51) Int. Cl. A61K 31/551; A61K 31/519; A61K 31/496; A61K 31/445

(52) U.S. Cl. 514/220; 514/252.16; 514/259.41; 514/262.1; 514/317; 514/254.06

(57) ABSTRACT

The use of phosphodiesterase 5 (PDE5) inhibitors for treatment of schizophrenia is described. Suitable PDE5 inhibitors for use for treatment of schizophrenia include sildenafil, vardenafil, tadalafil, E-8010, zaprinast, and E-4021. In one embodiment, for example, a method is described for treating schizophrenia in a patient which comprises treating the patient with an effective amount of a PDE5 inhibitor, or a pharmaceutically acceptable salt, solvate, or composition thereof. The PDE5 inhibitor may be administered orally. The PDE5 inhibitor may also be administered together with one or more conventional antipsychotic medications such as risperidone, olanzapine, quetiapine, ziprasidone, aripiprazole, clozapine, haloperidol, and fluphenazine.

### CROSS REFERENCE TO RELATED APPLICATIONS

The present application is related to and claims the benefit of priority of the following commonly-owned, presently-pending provisional application(s): application Ser. No. 60/671,198 (Docket No. OAK/0001.00), filed Apr. 14, 2005, entitled “Use of Phosphodiesterase 5 (PDE5) Inhibitors in the Treatment of Schizophrenia”, of which the present application is a non-provisional application thereof. The disclosure of the foregoing application is hereby incorporated by reference in its entirety, including any appendices or attachments thereof, for all purposes.

### COPYRIGHT NOTICE

A portion of the disclosure of this patent document contains material which is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### BACKGROUND OF THE INVENTION

1. Field of the Invention

The present invention relates to the use of phosphodiesterase type five (PDE 5) inhibitors for the treatment of schizophrenia.

2. Description of the Background Art

Schizophrenia is one of the most severe and debilitating of the major psychiatric diseases. It usually starts in late adolescence or early adult life and often becomes chronic and disabling. Men and women are at equal risk of developing this illness; however, most males become ill between 16 and 25 years old, while females develop symptoms between 25 and 30. Schizophrenia affects approximately 1% of the world population. The disease places a heavy burden on the patient's family and relatives, both in terms of the direct and indirect costs involved and the social stigma associated with the illness, sometimes over generations. Such stigma often leads to isolation and neglect.

Schizophrenia has traditionally been classified as a major psychotic disorder. The term psychotic denotes a loss of reality testing, which can occur as a result of delusional beliefs or hallucinatory perceptions, usually auditory or visual. The psychotic symptoms are the most dramatic and potentially dangerous features of this illness, but other symptoms may be even more disabling. The hallmark of paranoid schizophrenia is a delusional system in which unrelated and often bizarre ideas are linked. In schizophrenia patients, factor analysis has identified at least 3 symptom clusters that may vary independently over time. In addition to the psychotic symptom cluster of delusions and hallucinations, a second symptom cluster consists of disorganized thinking and behavior and inappropriate affect. A third cluster includes the negative symptoms of apathy, social withdrawal, loss of emotional expressiveness, and poverty of thought and speech. Persistent negative symptoms that are not attributable to depression, psychosis, or adverse medication effects make up the deficit syndrome. Patients with the deficit syndrome are often particularly unresponsive to treatment, and their amotivational state leaves them isolated and with poor rehabilitation potential. Deficits in attention, memory, and executive functions, although most prominent in patients with deficit syndrome, are present in most patients with schizophrenia and contribute substantially to difficulties with social interactions and vocational functioning.

Deficits in attention and memory are often detected only with formal testing. To meet diagnostic criteria for schizophrenia as established by the Diagnostic and Statistical Manual of Mental Disorders, Fourth Edition (Washington, D.C., American Psychiatric Association, 1994), an individual must demonstrate a decline in academic, vocational, or social functioning, or, if the illness develops in childhood, a failure to achieve an expected level of development in these areas. An active phase of the illness, consisting of a combination of psychotic and negative symptoms, must be present for at least 4 weeks unless successfully treated. The diagnosis of schizophrenia is not made until the illness—the prodromal decline in functioning and active-phase emergence of psychotic symptoms—has persisted for 6 months. Schizophrenia is a diagnosis of exclusion. Identifiable organic etiologies (for example, substance-induced psychotic states, endocrinopathies, structural or infectious brain lesions, and seizure disorders) must be ruled out. Other psychotic disorders must also be excluded, such as bipolar disorder or depression with psychotic features. The distinction between affective psychoses and schizophrenia can be difficult to identify early in the course of the illness, when affective symptoms may coexist with schizophrenia. Often, it is only by history obtained from family members and observation over time that clinicians can reliably identify the continuous decrement in functioning and persistence of psychotic and negative symptoms that characterize schizophrenia. A similar process of longitudinal observation may be necessary to determine that symptoms are not primarily the result of illicit substance use.

Although the content of delusions and auditory hallucinations may be culturally influenced, the incidence of schizophrenia is relatively uniform across cultures. The distribution of the illness, however, may be uneven both temporally and geographically. Several studies have found significant correlations between the size of the town or city of one's birth and subsequent risk of schizophrenia. Consistent, although modest, increases in the incidence of schizophrenia have been found in urban, industrialized areas. Whether the increased risk associated with an urban birth reflects migratory patterns vs exposure to viruses, toxins, or stress remains to be clarified. Several environmental insults occurring in utero or pennatally also increase the risk for schizophrenia. Although environmental factors can account for a considerable percentage of population-attributable risk, a complex genetic component accounts for the largest RR. Despite the strong hereditary component of the illness, attempts to establish genetic linkages have been largely disappointing. Several chromosomal regions have been identified in linkage studies, but none account for a substantial portion of variance. Most schizophrenia is sporadic, without affected first-degree relatives, although subtle neurocognitive deficits may be present in some unaffected relatives.

Increasingly, schizophrenia has been viewed as a neurodevelopmental disorder in which clinical symptoms emerge as brain maturation activates aberrant networks (see e.g., DeLisi LE “Is schizophrenia a lifetime disorder of brain plasticity, growth and aging?” Schizophr Res. 23.119-129 (1997)). However, the finding of progressive loss of brain volume in a subgroup of patients with poor outcomes suggests that an additional neurodegenerative process may be involved (see e.g., Lieberman J et al “Longitudinal study of brain morphology in first episode schizophrenia”, Biol Psychiatry. 49: 487-499 (2001)). Although most individuals destined to develop schizophrenia appear to be within the normal range for cognitive functioning and behavior in childhood, mean scores on IQ tests have been shown to be lower, and neuromotor impairment has been shown to be more common than in unaffected children.

Negative symptoms and cognitive deficits tend to persist relatively unchanged over time, whereas psychotic symptoms are variably responsive to pharmacotherapy. The initial response to medication best predicts longer-term functioning, as do the presence of cognitive deficits and negative symptoms. Most first-episode patients respond to conventional neuroleptics, with resolution of psychotic symptoms, but deterioration in functioning and repeated relapses tend to be the rule. Despite achieving full remission of psychotic symptoms for 1 year, 78% of first-episode schizophrenia patients in one recent study relapsed within 1 year after stopping medication; 96%, within 2 years (see e.g., Gitlin M et al “Clinical outcome following neuroleptic discontinuation in patients with remitted recent-onset schizophrenia”, Am J Psychiatry, 158: 1835-1842 (2001)). Schizophrenia patients may be at increased risk for committing violence, particularly if they are untreated and experiencing paranoid delusions that compel them to protect themselves. Another concern is the increased suicide rate of schizophrenia patients.

Comorbid conditions, such as depression and substance abuse, are common in schizophrenia and are associated with worse outcomes. Alcohol, cannabis, and stimulants are the substances most commonly abused by schizophrenia patients. Schizophrenia patients tend to underreport their own substance use and stimulants, alcohol, and cannabis can all worsen psychotic symptoms and often trigger relapse. Schizophrenia patients may use stimulants in part to improve prefrontal cortical functioning, as evidenced by improved attention and decreased negative symptoms. However, this potential benefit of stimulant use comes at a high price; a pattern of psychotic exacerbation and relapse. Similarly, cigarette smoking may be strongly reinforced by the therapeutic effects of nicotine upon certain attentional deficits in schizophrenia patients.

No single pathophysiologic mechanism has been identified that can account for the genetic vulnerability, the contribution of environmental risk factors, the delayed developmental onset, and a wide range of relatively subtle neuropathological findings, nor does any neuropathological finding reliably differentiate brains of patients with schizophrenia from those of healthy individuals. However, reduced brain volume has been consistently reported, with enlarged lateral and third cerebral ventricles and decreased volume of several brain structures, most notably the medial temporal lobe (see e.g., Wright IC et al “Meta-analysis of regional brain volumes in schizophrenia”, Am J Psychiatry, 157: 16-25 (2000)). Early functional neuroimaging studies identified hypoactivation of prefrontal cortex, but more recent work suggests aberrant activation of a wider network involving temporal and parietal cortices, striatum, thalamus, and cerebellum (see e.g., Andreasen N C et al “Hypofrontality in schizophrenia: distributed dysfunctional circuits in neuroleptic-naive patients” Lancet 349: 1730-1734 (1997)). Dysfunction of these cortical and subcortical networks presumably may result from a variety of lesions, and the extent of involvement may differ between subgroups of patients.

Postmortem examination has revealed relatively subtle changes in several areas of the brain and an absence of gliosis, suggesting that degenerative processes do not play a major role (see e.g., Goff D C et al “Schizophrenia”, Med Clin North Am, 85:663-689 (2001)). Akbarian and colleagues demonstrated abnormal distribution of a subset of pyramidal neurons in the frontal and temporal lobes consistent with abnormal neuronal migration during brain development (see e.g., Akbarian S et al “Altered distribution of nicotinamide-adenine dinucleotide phosphate-diaphorase cells in frontal lobe of schizophrenics implies disturbances of cortical development”, Arch Gen Psychiatry, 50: 169-177, (1993); and Akbarian S et al “Distorted distribution of nicotinamide-adenine dinucleotide phosphate-diaphorase neurons in temporal lobe of schizophrenics implies anomalous cortical development”, Arch Gen Psychiatry, 50: 178-187 (1993)). Increasingly, attention has focused on a loss of inhibitory interneurons, particularly in prefrontal cortex (see e.g., Pierri J N et al “Alterations in chandelier neuron axon terminals in the prefrontal cortex of schizophrenia subjects”, Am J Psychiatry. 156: 1709-1719 (1999)) and in the CA2 region of the hippocampus (see e.g., Benes F M et al “A reduction of nonpyramidal cells in sector CA2 of schizophrenics and manic depressives”, Biol Psychiatry, 44: 88-97 (1998)). The recent application of DNA microarray analysis to postmortem prefrontal cortical tissue revealed down-regulation of 6 genes involved in myelination by oligodendrocytes and up-regulation of several genes involved in synaptic plasticity, neuronal development, neurotransmission, and signal transduction compared with controls (see e.g., Hakak Y et al “Genome-wide expression analysis reveals dys-regulation of myelination-related genes in chronic schizophrenia” Proc Natl Acad Sci, USA, 98: 4746-4751 (2001)).

The dominant neurochemical model for schizophrenia has been the hyperdopaminergic hypothesis, based largely on the psychotic effects of high doses of dopamine agonists, such as amphetamine, and the antipsychotic effects of dopamine D2 receptor blockers (see e.g., Bell D S “The experimental reproduction of amphetamine psychosis” Arch Gen Psychiatry: 29:35-40 (1973); and Seeman P et al “Antipsychotic drug doses and neuroleptic dopamine receptors”, Nature, 261: 717-719 (1976)). A revision of this model posits diminished dopamine activity in the prefrontal cortex underlying negative symptoms and reciprocal dopaminergic hyperactivity in mesolimbic pathways responsible for psychotic symptoms (see e.g., Davis K L et al “Dopamine in schizophrenia: a review and reconceptualization”, Am J Psychiatry, 148: 1474-1486 (1991)). An early report of reduced D2 receptor density in the caudate of medication-naive schizophrenia patients has not been replicated consistently, although a preliminary finding of reduced D, receptor density in prefrontal cortex has been linked to negative symptoms and impaired working memory (see e.g., Okubo Y et al “Decreased prefrontal dopamine D1 receptors in schizophrenia revealed by PET” Nature, 385: 634-636 (1997)). Several investigators have demonstrated an abnormal increase in dopamine release in the caudate in response to amphetamine infusion (see e.g., Laruelle M et al “Single photon emission computerized tomography imaging of amphetamine-induced dopamine release in drug-free schizophrenic subjects”, Proc Natl Acad Sci USA, 93: 9235-9240 (1996)). One recent study showed response of schizophrenia patients as a result of dipyridamole on adenosine-dopamine receptor interactions (see e.g., Akhondzadeh S et al “Dipyridamole in the treatment of schizophrenia: adenosine-dopamine receptor interactions”, J Clin Pharm Ther 25(2):131-7 (2000)).

More recently, attention has been directed to glutamatergic systems in schizophrenia (see e.g., Goff D C, Coyle J T “The emerging role of glutamate in the pathophysiology and treatment of schizophrenia” Am J Psychiatry, 158: 1367-1377 (2001)), in part because of relatively consistent findings of altered glutamatergic receptor density and subunit composition in prefrontal cortex, thalamus, and temporal lobe. In addition, the non-competitive N-methyl-D-aspartate antagonists, phencyclidine (PCP) and ketamine, produce a compelling pharmacologic model of schizophrenia, which can include characteristic psychotic, negative, and cognitive symptoms (see e.g., Javitt D C, Zukin S R “Recent advances in the phencyclidine model of schizophrenia”, Am J Psychiatry, 148: 1301-1308 (1991)).

Before the introduction of chlorpromazine in 1953, most individuals with schizophrenia were destined to spend their entire adult lives within large, often remote psychiatric hospitals. Chlorpromazine and subsequent dopamine D2 receptor antagonists, or conventional neuroleptics, control most psychotic symptoms, allowing a majority of patients to live in the community. Negative symptoms and cognitive deficits remain largely unimproved, however. Blockade of D2 receptors produces hyperprolactinemia and a number of adverse neurological effects, including pseudoparkinsonism, motor restlessness (akathisia), and the potentially irreversible choreiform movements known as tardive dyskinesia. These adverse effects are often distressing to patients, and the behavioral manifestations can exacerbate stigmatization. Typical therapeutic doses of conventional neuroleptics produce hyperprolactinemia and adverse extrapyramidal effects in most patients, although careful dose adjustment can usually minimize extrapyramidal symptoms (see e.g., Baldessarini R J et al “Significance of neuroleptic dose and plasma level in the pharmacological treatment of psychoses”, Arch Gen Psychiatry, 45: 79-91 (1988)). Tardive dyskinesia occurs with a frequency of approximately 5% per year of exposure to conventional neuroleptics and is not dose-related. Elderly individuals have much higher rates of adverse neurological effects, including a 30% incidence of tardive dyskinesia during the first year of exposure. These side effects are so troublesome that many patients simply refuse to take the drugs. Thus, despite the beneficial effects of neuroleptics, even some patients who have a good short-term response will ultimately deteriorate in overall functioning. The well known deficiencies in the standard neuroleptics have stimulated a search for new treatments and have led to a new class of drugs termed atypical neuroleptics.

The first atypical neuroleptic, Clozapine, is effective for some patients who do not respond to standard neuroleptics. It also seems to reduce negative as well as positive symptoms, or at least exacerbates negative symptoms less than standard neuroleptics do. Clozapine does not cause tardive dyskinesia or extrapyramidal symptoms and is more effective than the previous generation of agents, producing a response in 30% to 50% of patients refractory to conventional antipsychotics. The range of clozapine's clinical benefits is broad compared with that of the conventional agents and includes enhanced efficacy for psychotic, negative, and affective symptoms, in addition to improved prophylaxis against relapse and violent behavior (see e.g., Rosenheck R et al “A comparison of clozapine and haloperidol in hospitalized patients with refractory schizophrenia”, N Engl J. Med. 337: 809-815 (1997); and Frankle W et al “Clozapine associated reduction in arrest rates of psychotic patients with criminal histories”, Am J Psychiatry, 158: 270-274 (2001)). It also has beneficial effects on overall functioning and may reduce the chance of suicide in schizophrenic patients. However, clozapine has serious limitations. It can cause agranulocytosis, a potentially lethal inability to produce white blood cells. Agranulocytosis remains a threat that requires careful monitoring and periodic blood tests. Clozapine can also cause seizures and other disturbing side effects (e.g., drowsiness, lowered blood pressure, drooling, bed-wetting, and weight gain). Thus it is usually taken only by patients who do not respond to other drugs.

Four atypical antipsychotic agents have followed clozapine and share a reduced propensity to produce adverse neurological effects (see e.g., Kapur S, Remington G “Atypical antipsychotics: new directions and new challenges in the treatment of schizophrenia”, Annu Rev Med., 52: 503-517 (2001)). Olanzapine and risperidone have been associated with greater efficacy for negative symptoms compared with haloperidol (see e.g., Marder S et al “The effects of risperidone on the five dimensions of schizophrenia derived by factor analysis: combined results of the North American trials”, J Clin Psychiatry, 58: 538-546 (1997); and Tollefson G D, Sanger™ “Negative symptoms a path analytic approach to a double-blind, placebo- and haloperidol-controlled clinical trial with olanzapine”, Am J Psychiatry, 154: 466-474 (1997)). The atypical agents may also improve cognitive functioning, but it is unclear whether any of the new atypical antipsychotics are equal to clozapine in overall efficacy. The reduction in adverse-effect burden with the newer agents has been associated with improved compliance and lowered hospitalization rates compared with that of conventional agents. Although only moderate enhancement of compliance has been demonstrated with the atypical agents (see e.g., Dolder C R et al “Antipsychotic medication adherence is there a difference between typical and atypical agents?”, Am J Psychiatry 159: 103-108 (2002)), randomized double-blind trials comparing risperidone (see e.g., Csernansky J G et al “A comparison of risperidone and haloperidol for the prevention of relapse in patients with schizophrenia”, N Engl J. Med., 346: 16-22 (2002)) and olanzapine (see e.g., Tran P et al “Oral olanzapine versus oral haloperidol in the maintenance treatment of schizophrenia and related psychoses”, Br J Psychiatry, 172: 499-505 (1998)) with haloperidol have found reductions in relapse rates of 43% and 30%, respectively. The reduction in adverse neurological effects with atypical agents has been attributed to additional antagonist activity at the serotonin 5-hydroxytryptamine (5HT2A) receptor, although recent evidence suggests that the newer agents may reduce adverse neurological effects in part by producing lower levels of sustained D2 receptor occupancy (see e.g., Kapur S, Seeman P, “Does fast dissociation from the dopamine D2 receptor explain the action of atypical antipsychotics? a new hypothesis”, Am J Psychiatry, 158: 360-369 (2001)). Although these newer agents may not produce some of clozapine's most troubling side effects, including agranulocytosis, there still have some side effects. For example, patients taking olanzapine may become sedated or dizzy, develop dry mouth, gain weight, or, in rare cases, liver function tests become transiently abnormal. Schizophrenia patients are more likely to be heavy cigarette smokers and frequently obese. Weight gain is a class effect of the atypical agents (with the exception of ziprasidone), although there is considerable variability between agents and between individual patients. A recent naturalistic study found a mean weight gain of 0.54 kg/mo with clozapine, which was not dose-related and did not plateau until a mean weight gain of approximately 9 kg was reached at 4 years (see e.g., Henderson D et al. “Clozapine, diabetes mellitus, weight gain, and lipid abnormalities: a five year naturalistic study”, Am J Psychiatry, 157: 975-981 (2000)). In addition, a considerable percentage of patients developed treatment-emergent diabetes mellitus during the first 5 years of clozapine treatment.

A more effective solution for treatment of schizophrenia without some of the side effects of existing treatments would be of considerable benefit. The cost of schizophrenia to society is enormous. Schizophrenia is estimated to account for about one fourth of all mental health costs and takes up one in three psychiatric hospital beds. Standardized mortality ratios (SMRs) for schizophrenic patients are estimated to be two to four times higher than the general population, and their life expectancy overall is 20% shorter than for the general population. The most common cause of death among schizophrenic patients is suicide (in 10% of patients) which represents a 20 times higher risk than for the general population. Deaths from heart disease and from diseases of the respiratory and digestive system are also increased among schizophrenic patients.

### GLOSSARY

The following definitions are offered for purposes of illustration, not limitation, in order to assist with understanding the discussion that follows.

“Cognitive impairment” refers to an acquired deficit in one or more of memory function, attention, problem solving, orientation and/or abstraction that impinges on an individual's ability to function independently.

“Dementia” refers to a global deterioration of intellectual functioning in clear consciousness, and is characterized by one or more symptoms of disorientation, impaired memory, impaired judgment, and/or impaired intellect. The symptoms of “dementia” are generally worse than, and can encompass, the symptoms of “cognitive impairment.”

“Patient” refers to animals, preferably mammals, more preferably humans. The term “patient” includes adults and children, and includes men and women. Children includes neonates, infants, and adolescents.

“PDE 5 inhibitors” refers to cyclic guanosine 3′,5′-monophosphate type five cGMP PDE 5 inhibitors (or phosphodiesterase 5 (PDE5) inhibitors) which are sometimes referred to herein as PDE V or PDE5 inhibitors. Suitable PDE5 inhibitors for use according to the present invention include sildenafil, tadalafil and vardenafil. These three PDE5 inhibitors are currently approved for the treatment of erectile dysfunction. PDE5 inhibitors increase cyclic guanosine monophosphate (cGMP) levels, which produces a neuroprotective effect via protein kinase 1 (PKG1) activation and enhances long-term potentiation.

“Schizophrenia” is a psychotic disorder characterized by impaired reality testing caused by delusions and hallucinations, and extensive withdrawal of the patient's interest from other people and the outside world, and the investment of it in his own. Patients diagnosed with schizophrenia often have cognitive impairments and/or dementia caused by the underlying disease process and/or as a side-effect of the treatments with antipsychotic medications. As used herein, the term “schizophrenia” refers to a psychiatric disorder that includes at least two of the following: delusions, hallucinations, disorganized speech, grossly disorganized or catatonic behavior, or negative symptoms. Patients can be diagnosed as schizophrenic using the DSM-IV criteria (APA, 1994, Diagnostic and Statistical Manual of Mental Disorders (Fourth Edition), Washington, D.C.).

### SUMMARY OF THE INVENTION

The present invention provides for the use of phosphodiesterase 5 (PDE5) inhibitors for treatment of schizophrenia. Suitable (PDE5) inhibitors for use according to the present invention include sildenafil, vardenafil, tadalafil, E-8010, Zaprinast, and E-4021. In one embodiment, for example, a method of the present invention is described for treating schizophrenia in a patient which comprises treating the patient with an effective amount of a phosphodiesterase type 5 (PDE5) inhibitor, or a pharmaceutically acceptable salt, solvate, or composition thereof. The PDE5 inhibitor may be administered orally and may be administered together with one or more conventional antipsychotic medications such as risperidone, olanzapine, quetiapine, ziprasidone, aripiprazole, clozapine, haloperidol, and fluphenazine.

In another embodiment, for example, the use of a phosphodiesterase type 5 (PDE5) inhibitor for manufacture of a medicament for treating schizophrenia is described.

In yet another embodiment, for example, a method of the present invention is described for treatment of schizophrenia in a mammal by administering a cyclic guanosine 3′,5′-monophosphate phosphodiesterase type five (cGMP PDE V) inhibitor, or a pharmaceutically acceptable salt, solvate, or composition thereof. The cGMP PDE V inhibitor may be administered orally. Preferably, it may also be administered together with one or more conventional antipsychotic medications such as risperidone, olanzapine, quetiapine, ziprasidone, aripiprazole, clozapine, haloperidol, and fluphenazine.

### BRIEF DESCRIPTION OF THE DRAWINGS

FIG. 1 is a high-level block diagram illustrating the manner in which PDE5 inhibitors are believed to enhance memory and learning via facilitation of long term potentiation (LTP) in more detail.

FIGS. 2A-F illustrate structures of the PDE5 inhibitors sildenafil (Viagra), vardenafil (Levitra), tadalafil (Cialis), E-8010, Zaprinast, and E-4021, respectively.

### DETAILED DESCRIPTION OF A PREFERRED EMBODIMENT

I. Overview

The present invention addresses the problem of inadequate pharmacological treatments for schizophrenia. Current treatments are relatively ineffective for cognitive impairments of schizophrenia—a problem that the National Institute of Mental Health (NIMH) has identified as a major priority for treatment development. A number of antipsychotic drugs are currently available for the treatment of schizophrenia. None are adequately effective, particularly for negative and cognitive symptoms. Current treatments are also relatively ineffective for negative symptoms and are effective for psychotic symptoms in roughly 70% of patients. No treatment has been shown to prevent progression of the illness, which may reflect neurotoxicity. Current treatments also have various other adverse side effects and limitations. Approximately 1% of the population suffers from schizophrenia and almost all of these individuals could potentially benefit from a drug that improves the characteristic cognitive deficits of the illness. Whereas treatments targeting dopamine receptors are effective for psychosis, a new model based on dysregulation of glutamatergic NMDA receptor activation better accounts for negative symptoms and cognitive deficits of schizophrenia (see e.g., Goff D C, Coyle J T: “The emerging role of glutamate in the pathophysiology and treatment of schizophrenia”, Am J Psychiatry 158:1367-1377 (2001)). This model is based in part on the observation that NMDA antagonists produce psychotic, negative, and cognitive symptoms in healthy subjects and recurrence of symptoms in stabilized patients with schizophrenia (see e.g., Javitt D, Zukin S, “Recent advances in the phencyclidine model of schizophrenia. Am J Psychiatry 1991 148:1301-1308 (1991)). Enhancement of NMDA activity by agonists at the glycine recognition site improved negative symptoms in some (see e.g., Goff D C et al “A placebo-controlled trial of D-cycloserine added to conventional neuroleptics in patients with schizophrenia”, Arch Gen Psych 1999 56:21-27 (1999)) but not all trials in schizophrenia patients (see e.g., Goff D C et al “A six-month, placebo-controlled trial of D-cycloserine added to conventional antipsychotics in schizophrenia patients”, Psychopharmacol in press). Single dose administration of the glycine site agonist, D-cycloserine, improves learning and memory in animal models (see e.g., Goff D C et al “Glutamatergic strategies for cognitive impairment in schizophrenia”, Psychiatr Ann 29:649-6548 (1999)); however, tolerance for memory effects develops after two weeks of daily dosing (see e.g., Quartermain D et al “Acute but not chronic activation of the NMDA-coupled glycine receptor with d-cycloserine facilitates learning and retention”, Eur J Pharm 257:7-12 (1994)). An 8-week trial of D-cycloserine failed to improve cognitive deficits in schizophrenia patients (see e.g., Goff D C et al “A placebo-controlled trial of D-cycloserine added to conventional neuroleptics in patients with schizophrenia”, above).

Because tolerance appears to limit the therapeutic potential of NMDA agonists, one strategy is to target pharmacological treatments at points “down stream” from the NMDA receptor. Activation of NMDA receptors allows calcium influx into the cell, which binds to calmodulin and activiates neuronal nitric oxide (NO) synthetase, thereby increasing NO, which activates guanylate cyclase, increasing cyclic guanine monphosphate (cGMP). This “glutamate-NO-cGMP intracellular pathway” is believed to mediate long term potentiation and memory consolidation (see e.g., Erceg S et al “Oral administration of sildenafil restores learning ability in rats with hyperammonemia and with portacaval shunts”, Hepatology 41(2):299-306 (2005); Prickaerts J et al “cGMP, but not cAMP, in rat hippocampus is involved in early stages of object memory consolidation”, Eur J Pharmacol 436(1-2):83-7 (2002); and Yamada K et al “Role of nitric oxide and cyclic GMP in the dizocilpine-induced impairment of spontaneous alternation behavior in mice”, Neuroscience 74(2):365-74 (1996)). Phosphodiesterase 5 (PDE5) inhibitors act to selectively increase cGMP without directly affecting NMDA receptors or NO. Targeting PDE5 to increase cGMP may selectively correct deficits resulting from NMDA receptor hypofunction and possibly avoid the problem of tachyphylaxis.

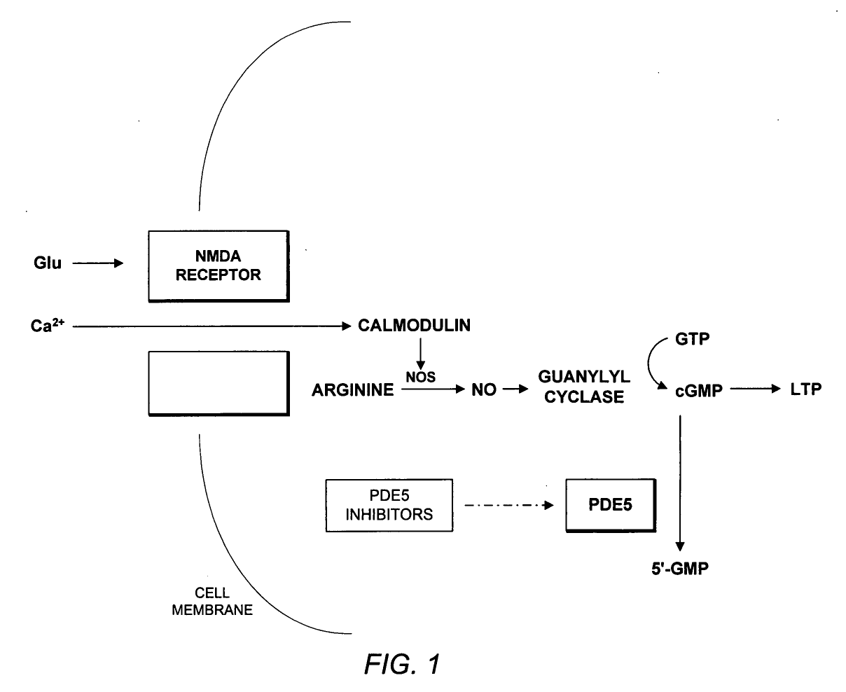

Phosphodiesterase 5 (PDE5) inhibitors are believed to enhance memory and learning via facilitation of long-term potentiation (LTP) mediated by the “glutamate-nitric oxide-cyclic GMP intracellular pathway”. FIG. 1 is a high-level block diagram illustrating the manner in which PDE5 inhibitors are believed to enhance memory and learning via facilitation of long term potentiation (LTP) in more detail. As shown, Gllutamate binding to the N-methyl-D-aspartate (NMDA) receptor results in calcium (Ca2+) influx, which binds to calmodulin and activates nitric oxide synthetase (NOS). NOS catalyzes the production of nitric oxide (NO) from arginine; NO activates guanylyl cyclase which increases production of cyclic guanosine monophosphate (GMP) from guanosine triphosphate (GTP). Cyclic GMP mediates LTP and activates protein kinases (PK) which are believed to mediate memory consolidation via phosphorylation and protein formation. PDE5 inhibitors block the conversion of cGMP to 5′GMP; by elevating cGMP levels, LTP is facilitated.

U.S. Pat. No. 6,469,012 (originally published as WO9428902 based on an initial UK filing), the disclosure of which is hereby incorporated by reference, discloses that compounds which are phosphodiesterase 5 (PDE5) inhibitors are potent and effective compounds for the treatment of male erectile dysfunction (MED, impotence) and for female sexual disorders. The discovery that PDE 5 inhibitors are effective for treatment of MED has led to the development of the compounds sildenafil, tadalafil, and vardenafil. Sildenafil, also known as VIAGRA™, and its pharmaceutically acceptable salts are described in U.S. Pat. No. 5,250,534, the disclosure of which is hereby incorporated by reference. Vardenafil hydrochloride, also known as LEVITRA™, is described in more detail in U.S. Pat. No. 6,683,080, the disclosure of which is hereby incorporated by reference. Tadalafil, also known as CIALIS™, is described in more detail in U.S. Pat. No. 6,140,329, the disclosure of which is hereby incorporated by reference.

It has now been found that phosphodiesterase 5 (PDE5) inhibitors have utility in the treatment of schizophrenia. The present invention therefore provides for the use of phosphodiesterase 5 (PDE5) inhibitors in the manufacture of a medicament for treatment of schizophrenia. Suitable PDE5 inhibitors for use according to the present invention include sildenafil, tadalafil and vardenafil, which are three PDE5 inhibitors currently approved for the treatment of erectile dysfunction (MED). Thus, the present invention provides for the use of PDE5 inhibitors, and in particular sildenafil, tadalafil and vardenafil, or a pharmaceutically acceptable salt thereof, or a pharmaceutical composition containing one of these compounds, for the manufacture of a medicament for the curative or prophylactic treatment of schizophrenia in a mammal, including a human. For human use, these compounds can be administered alone, but will generally be administered in admixture with a pharmaceutical carrier selected with regard to the intended route of administration and standard pharmaceutical practice. For example, the compound may be administered orally, buccally or sublingually, in the form of tablets containing excipients such as starch or lactose, or in capsules or ovules either alone or in admixture with excipients, or in the form of elixirs or suspensions containing flavouring or coloring agents. Such liquid preparations may be prepared with pharmaceutically acceptable additives such as suspending agents (e.g. methylcellulose, a semi-synthetic glyceride such as witepsol or mixtures of glycerides such as a mixture of apricot kernel oil and PEG-6 esters or mixtures of PEG-8 and caprylic/capric glycerides). For best treatment results the PDE 5 inhibitors are preferably used together with other standard (conventional) antipsychotic medications such as risperidone, olanzapine, quetiapine, ziprasidone, aripiprazole, clozapine, haloperidol and/or fluphenazine.

The present invention is unique in that the potential of PDE5 inhibitors as a treatment for schizophrenia has not previously been proposed. PDE5 inhibition represents a new target for drug treatment and potentially may improve symptoms which are not adequately responsive to existing treatments. The PDE5 inhibitors readily cross the blood brain barrier and produce cognitive and behavioral effects that suggest it may produce broad therapeutic effects in schizophrenia. PDE5 has been identified in rat brain, including the cortex and hippocampus (see e.g., van Staveren W C, et al “Localization and characterization of cGMP-immunoreactive structures in rat brain slices after NO-dependent and NO-independent stimulation of soluble guanylyl cyclase”, Brain Res 1036(1-2):77-89 (2005); and Van Staveren W C, et al “mRNA expression patterns of the cGMP-hydrolyzing phosphodiesterases types 2, 5, and 9 during development of the rat brain”, J Comp Neurol 467(4):566-80 (2003)), and subchronic administration of sildenafil has been demonstrated to increase cortical cGMP levels in rats (see e.g., Zhang R et al “Sildenafil (Viagra) induces neurogenesis and promotes functional recovery after stroke in rats”, Stroke 33(11):2675-80 (2002); and Prickaerts J et al “Effects of two selective phosphodiesterase type 5 inhibitors, sildenafil and vardenafil, on object recognition memory and hippocampal cyclic GMP levels in the rat”, Neuroscience 113(2):351-61 (2002)).

Intrahippocampal administration of an inhibitor of guanylate cyclase (to selectively lower cGMP concentrations) has been shown to impair learning of inhibitory avoidance in rats (see e.g., Bernabeu R et al “Hippocampal cGMP and cAMP are differentially involved in memory processing of inhibitory avoidance learning”, Neuroreport 7(2):585-8 (1996)), whereas administration of an analog of cGMP facilitated memory consolidation (Bernabeu R et al “Further evidence for the involvement of a hippocampal cGMP/cGMP-dependent protein kinase cascade in memory consolidation”, Neuroreport 8(9-10):2221-4 (1997)). Hyperammonemia inhibits the activation of gluanylate cyclase by NO, which is hypothesized to account for the cognitive deficits (encephalopathy) associated with liver failure. Chronic oral treatment for 28 days with sildenafil normalized cGMP levels in brains of hyperammonemic rats and restored performance on learning tasks (Erceg S et al “Oral administration of sildenafil restores learning ability in rats with hyperammonemia and with portacaval shunts”, Hepatology 41(2):299-306 (2005)).

Sildenafil 3 mg/kg and 10 mg/kg p.o. significantly improved retention at 24 hours of an object recognition task in rats (Prickaerts J et al “Effects of two selective phosphodiesterase type 5 inhibitors, sildenafil and vardenafil, on object recognition memory and hippocampal cyclic GMP levels in the rat” Neuroscience 113(2):351-61 (2002)) and sildenafil 3 mg/kg improved retention at 48 hours of a passive avoidance task utilizing an aversive stimulus (Baratti C M, et al “Effects of sildenafil on long-term retention of an inhibitory avoidance response in mice”, Behav Pharmacol 10(8):731-7 (1999)). Sildenafil 2, 4, & 8 mg/kg also dose-dependently improved acquisition of maze learning in mice (Singh N, et al “Sildenafil improves acquisition and retention of memory in mice”, Indian J Physiol Pharmacol 47(3):318-24 (2003)). PDE inhibitors improved performance in a spatial discrimination task but not in a test of spatial recognition memory (Prickaerts J et al “Phosphodiesterase type 5 inhibition improves early memory consolidation of object information”, Neurochem Int 45(6):915-28 (2004)). In a placebo-controlled trial in healthy young men, sildenafil 100 mg significantly affected event-related brain potentials (ERPs) patterns consistent with enhanced ability to focus attention and to select relevant target stimuli (Schultheiss D et al “Central effects of sildenafil (Viagra) on auditory selective attention and verbal recognition memory in humans: a study with event-related brain potentials”, World J Urol 19(1):46-50 (2001)) although actual performance did not significantly improve.

PDE 5 inhibitors are believed to protect against neurotoxicity, which has been hypothesized to play a role in the deteriorating course of some patients with schizophrenia (Goff D C et al “The emerging role of glutamate in the pathophysiology and treatment of schizophrenia”, above). Daily treatment for seven days with sildenafil 2-5 mg/kg following middle cerebral artery occlusion resulted in significantly greater functional recovery (foot fault test and adhesive removal test) and a greater cellular proliferation in the subventricular zone and dentate gyrus compared to vehicle as measured by bromodeoxyuridine labeling and b III tubulin staining for immature cells (Zhang R et al “Sildenafil (Viagra) induces neurogenesis and promotes functional recovery after stroke in rats”, above). PDE5 inhibitors also protected rat spinal neurons against toxicity from reactive oxygen species and chronic glutamate exposure (Nakamizo T et al “Phosphodiesterase inhibitors are neuroprotective to cultured spinal motor neurons”, J Neurosci Res 71(4):485-95 (2003)).

A single dose of sildenafil 1 mg/kg also enhanced behavioral effects of the dopamine agonists, 7-OH-DPAT and B-HT 920 in rats, whereas at 10 mg/kg it antagonized behavioral effects (Ferrari F et al “Influence of sildenafil on central dopamine-mediated behaviour in male rats”, Life Sci 70(13): 1501-8 (2002)). Low dopamine activity in prefrontal cortex has been postulated to contribute to cognitive deficits and negative symptoms of schizophrenia and enhancement of dopaminergic tone has been identified as a potential treatment for cognitive deficits in schizophrenia. Sidenafil has been reported to be safe and effective when administered to male schizophrenia patients for erectile dysfunction (Aviv A et al “An open-label trial of sildenafil addition in risperidone-treated male schizophrenia patients with erectile dysfunction”, J Clin Psychiatry 65(1): 97-103 (2004)). Phosphodiesterases (PDEs) and PDE inhibitors, and in particular PDE 5 inhibitors, are described below in more detail.

II. Detailed Operation

Introduction to Phosphodiesterases

Phosphodiesterases (PDEs) are a superfamily of enzymes that degrade cyclic adenosine monophosphate (cAMP) and cyclic guanosine monophosphate (cGMP) (Beavo J. A. “Cyclic nucleotide phosphodiesterases: functional implications of multiple isoforms”, Physiol. Rev., 75: 725-748 (1995), Soderling S. H. and Beavo J. A. “Regulation of cAMP and cGMP signaling: new phosphodiesterases and new functions” Curr. Opin. Cell Biol., 12: 174-179 (2000), Corbin J. D. and Francis, S. H. “Cyclic GMP phosphodiesterase-5: target of sildenafil”, J. Biol. Chem., 274: 13729-13732 (1999)). There are now 11 PDE families identified, many of which exist as splice variants (Beavo J A et al “Multiple cyclic nucleotide phosphodiesterases”, Mol. Pharmacol. 46: 399-3405 (1994), and Bolger G. B. “Molecular biology of the cyclic AMP-specific cyclic nucleotide phosphodiesterases: a diverse family of regulatory enzymes”, Cell. Signal. 6: 851-859 (1994)). The cAMP-specific enzymes include PDE4, -7 and -8. The cGMP-specific PDEs are PDE5, -6 and -9, whereas PDE1, -2, -3, -10 and -11 use both cyclic nucleotides (Mehats C et al “Cyclic nucleotide phosphodiesterases and their role in endocrine cell signaling”, Trends Endocr. Met. 13: 29-35 (2002)). PDEs influence a vast array of pharmacological processes, including proinflammatory mediator production and action, ion channel function, muscle contraction, learning, differentiation, apoptosis, lipogenesis, glycogenolysis and gluconeogenesis (Perry M. J. and Higgs G. A. “Chemotherapeutic potential of phosphodiesterase inhibitors”, Curr. Opin. in Chem. Biol. 2: 472-481 (1998)). As essential regulators of cyclic nucleotide signaling with diverse physiological functions, PDEs have become recognized as important drug targets for the treatment of various diseases, such as heart failure and erectile dysfunction (Rotella D. P. “Phosphodiestease 5 inhibitors: current status and potential applications” Nat. Rev. Drug Discov. 1: 674-682 9 (2002), Conti et al. “Recent progress in understanding the hormonal regulation of phosphodiesterases” Endocr. Rev. 16: 370-389 (1995), and Torphy T. J. “Phosphodiesterase isozymes”, Am. J. Respir. Crit. Care Med. 157: 351-370 (1998)).

cAMP and cGMP are ubiquitous second messengers responsible for transducing effects of various extracellular signals, including hormones, light and neurotransmitters. These cyclic nucleotides are formed from ATP and GTP by the catalytic reactions of adenylyl cyclase and guanylyl cyclase, respectively. Adenylyl cyclase can be activated by forskolin and guanylyl cyclase by nitric oxide (NO). Through cell-surface receptors such as b-adrenoreceptor and prostaglandin E2, these enzymes can also be activated indirectly (Torphy T. J. “Phosphodiesterase isozymes”, above). As the intracellular concentrations of the cyclic nucleotides rise, they bind to and activate their target enzymes, protein kinase A (PKA) and protein kinase G (PKG). These protein kinases phosphorylate substrates such as ion channels, contractile proteins and transcription factors, which regulate key cellular functions. Phosphorylation alters the activity of these substrates and thus changes cellular activity. Obviously, altering the rate of cyclic nucleotide formation or degradation will change the activation state of these pathways (Krebs E. G. and J. A. Beavo “Phosphorylationdephosphorylation of enzymes” Annu. Rev. Biochem. 48: 923-959 (1979)).

Kinetically distinct PDEs can be inhibited selectively by a variety of small organic molecules (Levin R. M. et al “Mechanism by which psychotropic drugs inhibit adenosine cyclic 39,59-monophosphate phosphodiesterase of brain”, Mol. Pharmacol. 12: 581-589 (1976), Hidaka H. et al “Selective inhibitors of three forms of cyclic nucleotide phosphodiesterases”, Trends Pharmacol. Sci. 5: 237-239 (1984), Wells J. N. et al “Inhibition of separated forms of cyclic nucleotide phosphodiesterase from pig coronary arteries by 1,3-disubstituted and 1,3,8-trisubstituted xanthines”, J. Med. Chem. 24: 954-958 (1981), and Wood M. A. et al “Review: long-term oral therapy of congestive heart failure with phosphodiesterase inhibitors”, Am. J. Med. Sci. 297: 105-113 (1989)). Recent advances in understanding the 3D structure of PDEs and their inhibitors facilitate rational drug discoveries and optimization of lead compounds. The 3D structures of the catalytic domains of PDE1, -3, -4, -5 and 9 are currently available. See e.g., Huai Q., et al “Crystal structures of phosphodiesterases 4 and 5 in complex with inhibitor 3-isobutyl-1-methylxanthine suggest a conformation determinant of inhibitor selectivity” J. Biol. Chem. 279: 13095-13101 (2004). Also see e.g., Sung B. J. et al “Structure of the catalytic domain of human phosphodiesterase 5 with bound drug molecules”, Nature 425: 98-102 (2003). 3D coordinates can be accessed to investigate binding of inhibitors, substrate discrimination mechanisms of PDEs, inhibitor selectivity and information on further optimization of inhibitors.

Structural Basis of PDE Catalysis and Inhibition

Various genes encoding human PDEs can be classified by their substrate specificities. One group of PDEs selectively hydrolyzes cyclic AMP (PDE4, -7 and -8), the second group of PDEs are cyclic GMP-specific enzymes (PDE5, 6 and -9), and the rest hydrolyze both cAMP and cGMP (PDE1, -2, -3, -10 and -11) (see e.g., Beavo J. A. and Brunton L. L. “Cyclic nucleotide research-still expanding after half a century”, Nat. Rev. Mol. Cell Biol. 3: 710-716 (2002); Conti M. “Phosphodiesterases and cyclic nucleotide signaling in endocrine cells”, Mol. Endocrinol. 14: 1317-1327 (2000); and Mehats C. et al “Cyclic nucleotide phosphodiesterases and their role in endocrine cell signaling”, Trends Endocrinol. Metab. 13: 29-35 (2002)). PDEs contain three functional domains, including a conserved catalytic core, a regulatory N-terminus, and the C-terminus (see e.g., Thompson W. J. “Cyclic nucleotide phosphodiesterases: pharmacology, biochemistry and function”, Pharmacol. Ther. 51: 13-33 (1991); and Bolger G. B. “Molecular biology of the cyclic AMP specific cyclic nucleotide phosphodiesterases: a diverse family of regulatory enzymes”, Cell. Signal. 6: 851-859 (1994)). Regulatory N-terminal domains of these enzymes that vary widely among the PDE classes are flanked by the catalytic core and include regions that auto-inhibit the catalytic domains, as well as targeting sequences that control subcellular localization (see e.g., Houslay M. D. et al “PDE4 cAMP phosphodiesterases: modular enzymes that orchestrate signaling cross-talk, desensitization and compartmentalization”, Biochem. J. 370: 1-18 (2003); and Sonnenburg W. K. et al “Identification of inhibitory and calmodulin-binding domains of the PDE1A1 and PDE1A2 calmodulin-stimulated cyclic nucleotide phosphodiesterases”, J. Biol. Chem. 270: 30989-31000 (1995)). This region contains a calmodulin binding domain in PDE1, cyclic GMP binding sites in PDE2, phosphorylation sites for various protein kinases in PDE1-5, and a transducin binding domain in PDE6. All PDEs contain a conserved catalytic domain of approximately 270 amino acids (18-46% of sequence identity) at the carboxyl terminus.

Due to the need to develop selective PDE inhibitors as therapeutic drugs, the structures of the catalytic domains of PDEs, which contain the active pocket that accommodates inhibitors, have been elucidated. The crystal structures of the catalytic domains of PDE4B, PDE4D, PDE5A, PDE3B, PDE1B, and PDE9A have shown that catalytic domains of PDEs have three helical subdomains. The catalytic domain of the PDE5 molecule can be divided into three subdomains: an N-terminal cyclin-fold domain (residues 537-678), a linker helical domain (residues 679-725) and a C-terminal helical bundle domain (residues 726-860)). As described by Sung B. et al. in “Structure of the catalytic domain of human phosphodiesterase 5 with bound drug molecules”, above, a surface representation of the active site of PDE5A occupied by sildenafil shows a deep hydrophobic pocket which is formed at the interface of the three subdomains and is composed of four subsites: a metal-binding site (M site), core pocket (Q pocket), hydrophobic pocket (H pocket) and lid region (L region). The M site is at the bottom of the pocket with several metal atoms, which bind to residues that are completely conserved in all PDE family members. Although the identity of the metal ions cannot be absolutely determined from the crystal structures, the observed geometry of the metal coordinating ligands, anomalous X-ray diffraction behavior and existing biochemical evidence all suggest that at least one of the metals is zinc and the other is likely to be magnesium (see e.g., Percival M. D. et al “Zinc dependent activation of cAMP-specific phosphodiesterase (PDE4A)”, Biochem. Biophys. Res. Commun. 241: 175-18044-47 (1997); Laliberte F. et al “Conformational difference between PDE4 apoenzyme and holoenzyme”, Biochemistry 39: 6449-6458 (2000); Kovala T. et al “Recombinant expression of a type IV, cAMP-specific phosphodiesterase: characterization and structure-function studies of deletion mutants”, Biochemistry 36: 2968-2976 (1997); and Francis S. H. et al “Zinc interactions and conserved motifs of the cGMP-binding cGMP-specific phosphodiesterase suggest that it is a zinc hydrolase”, J. Biol. Chem. 269: 22477-22480 (1994)). In the PDE structures, these metal ions have an octahedral coordination geometry. The zinc coordination sphere is made up of three histidines, one aspartate and two water molecules, while the magnesium coordination sphere involves the same aspartate and five water molecules, one of which is shared with the zinc molecule. The putative roles of these metal ions include stabilization of the structure and activation of hydroxide to mediate catalysis.

In the crystal structure of PDE5A in complex with sildenafil (Viagra), the Q pocket accommodates the pyrazolopyrimidinone group of sildenafil. This Q pocket provides the key hydrogen bonding of the conserved glutamine residue with substrates or inhibitors of PDEs, and the hydrophobic interactions, which come from the residues on both sides of the pyrazolopyrimidinone group, forming a “clamp” like structure. The ethoxyphenyl group of sildenafil fits into the hydrophobic H pocket. The variation of hydrophobic residues in the H pocket among PDEs can give PDE inhibitors the selectivity to corresponding PDEs. The L region of PDE5A, composed of residues Tyr 664, Met 816, Ala 823 and Gly 819, surrounds the methylpiperazine group of sildenafil. The conformational change between closed and open forms of this region seems to be involved in inhibitor binding. Structural features of each PDE have shown how the specificity of the substrate can be achieved. It has been proposed that PDE selectivity toward cyclic nucleotide is controlled by a so-called, “glutamine switch” mechanism (Zhang K. Y. et al “A glutamine switch mechanism for nucleotide selectivity by phosphodiesterases”, Mol. Cell. 15: 279-286 (2004)). It has been proposed that an invariant glutamine residue plays an important role in PDE nucleotide selectivity, but the structures reveal an invariant. The g-amino group of the conserved glutamine residue in the active site of the PDEs can alternatively adopt two different orientations: in one orientation the hydrogen bond network supports guanine binding, resulting in cGMP selectivity, and in the other orientation the network supports adenine binding, leading to selectivity toward cAMP. And in dual-specific PDEs the orientation of the side chain of lutamine can switch between the two orientations, resulting in dual specificity toward both cyclic nucleotides.

The crystal structures of the catalytic domains of PDEs in complex with several inhibitors are now known. The overall folding patterns of the catalytic domains of PDEs are very similar, with compact a-helical structures. However, the comparison of ligand binding sites to different PDE family members can aid in understanding what is common to ligand binding and what regions of inhibitors or drugs are important for selectivity for individual PDE family members. Common features in ligand binding of PDEs are as follows: The central rings of inhibitors on the position of the purine rings of cAMP or cGMP interact with the conserved glutamine by a bidentate or single H-bond. In the structure of PDE5A in complex with sildenafil, the pyrazolopyrimidinone group of sildenafil mimics that of guanine in cGMP and has the same H-bond donor and acceptor features to form a bidentate H-bond through its amide orientation evolved to bind cGMP. Another common characteristic is that the central rings of inhibitors are tightly held by a ‘hydrophobic clamp’ composed of side chains of hydrophobic residues. For example, the guanine moiety of cGMP or the pyrazolopyrimidinone group of sildenafil is sandwiched between the side chains of hydrophobic residues. Finally, in contrast to the substrate binding, PDE inhibitors are not involved in interaction with metal ions. Effective interaction with metals directly or indirectly via water molecules may improve the potency of inhibitors. Some side effects of sildenafil are known, and the main reason of the side effects is thought to be interaction with PDEs other than PDE5. To overcome the side effects of PDE inhibitors, the selectivity of inhibitors should be improved. Effective hydrophobic interaction of PDE5 inhibitors is crucial for the selective inhibition. These structural insights further facilitate the understanding of PDEs and the design of PDE inhibitors. PDE5 inhibitors will next be described in more detail.

PDE5 Inhibitors

A variety of physiological processes in the cardiovascular, nervous and immune systems are controlled by the NO/cGMP signaling pathway. In smooth muscle, NO and natriuretic peptides regulate vascular tone by inducing relaxation through cGMP (see e.g., Sausbier M. et al “Mechanisms of NO/ cGMP-dependent vasorelaxation”, Circ. Res. 87: 825-83068 (2000)). Degradation of cGMP is controlled by cyclic nucleotide PDEs, and PDE5 is the most highly expressed PDE that hydrolyzes cGMP in these cells. The physiological importance of PDE5 in regulation of smooth muscle tone has been demonstrated most clearly by clinical use of its specific inhibitors, sildenafil (Viagra), vardenafil (Levitra) and tadalafil (Cialis) in the treatment of erectile dysfunction (see e.g., Ballard S. A. et al “Effects of sildenafil on the relaxation of human corpus cavernosum tissue in vitro and on the activities of cyclic nucleotide phosphodiesterase isozymes”, J. Urol. 159: 2164-2171 (1998)). When a man is sexually stimulated, either physically or psychologically, NO is released from noncholinergic, nonadrenergic neurons in the penis, as well as from endothelial cells. NO diffuses into cells, where it activates soluble guanylyl cyclase, the enzyme that converts GTP to cGMP. cGMP then stimulates PKG, which initiates a protein phosphorylation cascade. This results in a decrease in intracellular levels of calcium ions, leading ultimately to dilation of the arteries that bring blood to the penis and compression of the spongy corpus-cavernosum tissue. This compression contracts veins, which reduces the outflow of blood and increases intracavernosal pressure, resulting in an erection (see e.g., Lue T. F. “Erectile dysfunction” N. Engl. J. Med. 324: 1802-1813 (2000)). A PDE5 inhibitor will retard enzymatic hydrolysis of cGMP in the human corpus cavernosum, leading to the same outcome. Sildenafil has also been demonstrated to improve sexual performance in women affected by arousal disorders in a double-blind, crossover and placebo-controlled study (Caruso S. et al “Premenopausal women affected by sexual arousal disorder treated with sildenafil: a double-blind, cross-over, placebocontrolled study” Br. J. Obstet. Gynecol. 108: 623-628 (2001)). However, there are some controversies for the evidence of efficacy of the PDE5 inhibitor for the treatment of female sexual dysfunction (FSD) (Segraves R. T. “Emerging therapies for female sexual dysfunction” Expert. Opin. Emerg. Drugs 8: 515-522 (2003)). For example, in the phase I trial of tadalafil, an orally active PDE5 inhibitor for the treatment of erectile dysfunction (ED), reported in June 2001, the results showed no conclusive treatment effect relative to placebo in women with FSD (IC351 shows no benefit over placebo in an exploratory female sexual arousal disorder trial, Lilly ICOS LLC press release posted on 18 Jun. 2001).

Given the multitude of cellular responses that cAMP and cGMP can elicit, it is clear that to achieve specificity of signal transduction, cells must be able to tightly regulate the magnitude and duration of cAMP/cGMP elevation, and also in specific cellular locations. Mammalian cells have evolved a complex and highly conserved complement of enzymes in order to generate, recognize and inactivate cyclic nucleotides. Inactivation of cAMP/cGMP is achieved by hydrolysis of the 3′-ester bond catalyzed by the PDEs, of which more than 50 have been identified (see e.g., Beavo J. A. “Cyclic nucleotide phosphodiesterase: functional implications of multiple isoforms” Physiol. Rev. 75: 725-748 (1995)). If cells did not possess PDEs, intracellular cAMP levels should rapidly become uniform. These enzymes therefore provide a key ability for the cell to generate nonuniform intracellular distribution of cAMP/cGMP, and hence differentially activate distinct compartmentalized protein kinase species.

PDE inhibitors reduce the hydrolysis of cAMP/cGMP, and hence elevate the intracellular level of cAMP/cGMP. Thus, PDE inhibitors will change the activation state of cyclic nucleotide signaling pathways, resulting in the regulation of various physiological functions. An important issue in the development of one PDE inhibitor is specificity for the other PDEs. PDE5 catalyzes the hydrolysis of cGMP with absolute specificity. The enzyme is active as a homodimer, which has a molecular mass of approximately 200 kDa. Either PKA or PKG can phosphorylate PDE5, and this results in a significant increase in PDE5 activity (see e.g., Corbin J. D. et al “Phosphorylation of phosphodiesterase-5 by cyclic nucleotide dependent protein kinase alters its catalytic and allosteric cGMP binding affinitys”, Eur. J. Biochem. 267: 2760-2767 (2000)). The protein is widely distributed throughout the smooth muscle in the body, and is also found in platelets (see e.g., Rotella D. P. “Phosphodiesterase 5 inhibitors: current status and potential applications” Nat. Rev. Drug Discov. 1: 674-684 (2002)). However, PDE5 exhibits a more limited tissue distribution than PDE1 and -2; it is particularly prevalent in vascular smooth muscle (see e.g., Yanaka N. “Expression, structure and chromosomal localization of the human cGMP-binding, cGMP specific phosphodiesterase PDE5 gene”, Eur. J. Biochem. 255: 391-3999 (1998)). PDE5 is the primary cGMP-hydrolyzing activity in human corpuscavernosum tissue. Erection is largely a hemodynamic event which is regulated by vascular tone and blood-flow balance in the penis. Because cGMP levels modulate vascular tone, PDE5 is an obvious target for therapeutic intervention in the process. Oral PDE5 inhibitors can increase the cGMP, smooth muscle relaxation in the penis and, thus, penis election. Similar mechanisms appear to be involved in genical vasodilatation in the human female (see e.g., Rosen R. et al “PDE-5 inhibition and sexual responses: pharmacological mechanism and clinical outcomes”, Annu. Rev. Sex Res. 13: 36-88 (2003)).

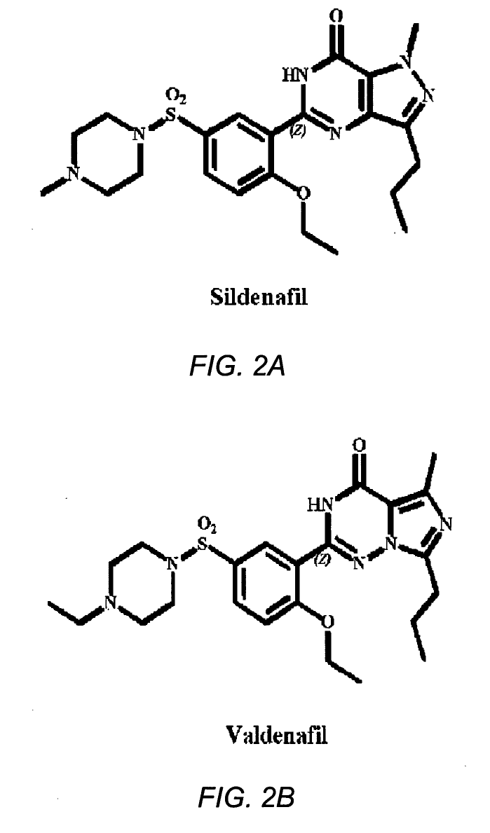

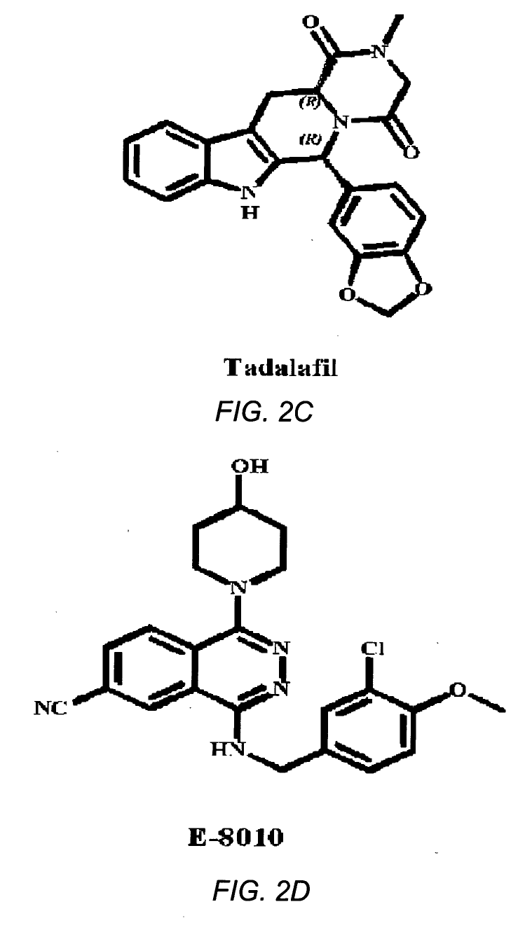

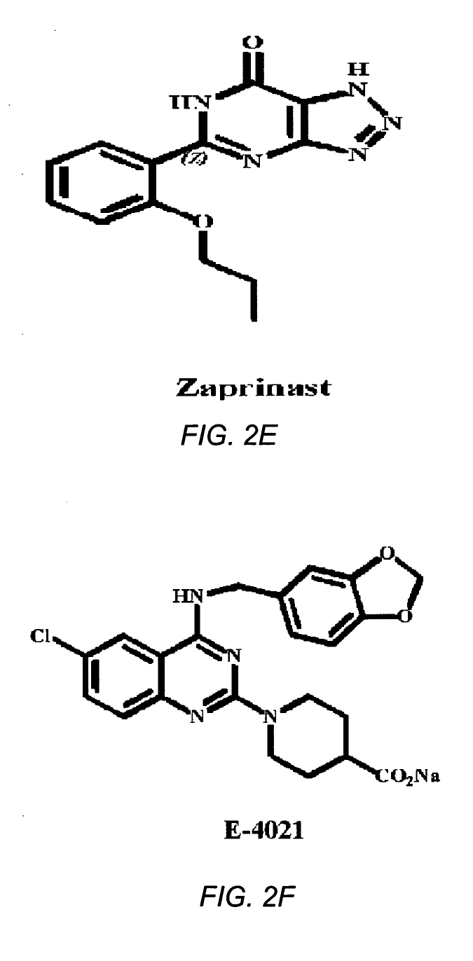

FIGS. 2A-F illustrate structures of the PDE5 inhibitors sildenafil (Viagra), vardenafil (Levitra), tadalafil (Cialis), E-8010, Zaprinast, and E-4021, respectively. Sildenafil, as shown at FIG. 2A, is an orally active, potent and selective inhibitor of cGMP-specific PDE5 (see e.g., Goldstein I. et al “Oral sildenafil in the treatment of erectile dysfunction”, N. Engl. J. Med. 338: 1397-1404 (1998); and Rosenberg K. P. “Sildenafil citrate for SSRI-induced sexual side effects”, Am. J. Psychiatry 156: 157 (1999)). Following oral administration, sildenafil is rapidly absorbed, with an absolute bioavailability of 40%. The time to peak plasma concentration (Tmax) after oral absorption in the fasting state has a range of 30-120 min, but a high-fat meal increases the Tmax by 60 min and reduces the peak plasma concentration by 29% (there is no effect on area under the curve [AUC]). From a clinical point of view, the onset of efficacy is optimal if sildenafil is taken on an empty stomach. The terminal half-life of sildenafil is 3-5 h (see e.g., Briganti A. et al. “Emerging oral drug for erectile dysfunction”, Expert Opin. Emerg. Drugs 9: 179-189 (2004)). Sildenafil was approved for use in the United States in March 1998. Because of its mechanism of action, sildenafil is contraindicated in patients taking NO donors or organic nitrates. The patient population with the greatest risk of developing ED comprises men over the age of 40. Many men in this age group also have other chronic diseases, such as depression, diabetes, atherosclerosis, hypertension or ischemic heart disease. All of these conditions increase the risk of developing ED, and in some cases, the pharmacological treatment for the disorder can also induce ED. Consequently, the safety and efficacy of sildenafil and other PDE5 inhibitors in this group of patients needed to be established. Several studies have been done with sildenafil in men with cardiovascular disease. The data indicate that, with the exception of patients taking organic nitrates, sildenafil does not have a synergistic effect on blood pressure with antihypertensive agents, such as ACE inhibitors, a-adrenoceptor or b-adrenoceptor blockers, calcium channel blockers or diuretics (see e.g., Kloner R. A. et al “Effect of sildenafil in patients with erectile dysfunction taking antihypertensive therapy” Am. J. Hypertens. 14: 70-73 (2001)). There was no increase in the incidence of drug-related adverse events, and the overall safety profile indicated that there was no significant difference in the incidence of stroke, myocardial infarction or other serious cardiovascular events in patients taking sildenafil. The drug improved erectile function in up to 70% of men with ischemic heart disease (see e.g., Conti R. C. et al “Efficacy and safety of sildenafil citrate in the treatment of erectile dysfunction in patients with ischemic heart disease”, Am. J. Cardiol. 83: 29C-34C (1999)), and gave similar results in trials with other groups of men with cardiovascular disease (see e.g., Angulo J. et al. “IC351 enhances NO-mediated relaxation of human arterial and trabecular penile smooth muscle”, Eur. Urol. 39: 106 (Abstract 415) (2001)).

Tadalafil, as shown at FIG. 2C, is another novel PDE5 inhibitor recently approved both in Europe and in the United States. It has a maximum Tmax of 2 h and a half-life of 17.5 h. The latter values clearly distinguish tadalafil from the other PDE5 inhibitors. When the selectivity profile of tadalafil was evaluated against 14 human recombinant PDEs, tadalafil was found to be highly selective for PDE5, with 700-fold greater affinity for PDE5 than for the related retinal PDE6 (see e.g., Angulo J., et al “IC351 enhances NO-mediated relaxation of human arterial and trabecular penile smooth muscle”, above). Furthermore, tadalafil has shown 14-fold greater affinity for PDE5 compared with PDEL11, which closely resembles PDE5 (71% amino acid similarity). Tadalafil also has a more rapid onset of action than sildenafil, often showing effects in 20 min or less (see e.g., Padma-Nathan H. et al “Cialis (IC351) provides prompt response and extended responsiveness for the treatment of erectile dysfunction”, J. Urol. 165: 224 (2001)). It is likely to be contraindicated in patients taking organic nitrates, in spite of a substantial increase in PDE5 selectivity compared with other PDE enzymes (see e.g., Porst H. IC351 “(tadalafil, Cialis): update on clinical experience” Int. J. Impotence Res. 14 (Suppl. 1): S57-S64 (2002)). In healthy subjects who received a single 20-mg dose, there was no significant change in heart rate, standing systolic or diastolic blood pressure. Analysis of the data from phase III clinical trials showed that the incidence of adverse events in patients taking tadalafil, including those with various cardiovascular diseases, was no different from that in placebo-treated patients (see e.g., Hutter A. M. “Blood pressure and cardiovascular effects of tadalafil, a new PDE5 inhibitor” Am. J. Hypertens, 15 (Part 2): 140A9 (2002)). In double-blind, placebo-controlled phase III trials that included over 1100 men, tadalafil doses of 2.5-20 mg once daily, as needed, significantly improved erections in up to 81% of men. The mean percentage of successful intercourse attempts was 75%, and efficacy was maintained in both hypertensive and nonhypertensive patient groups (see e.g., Padma-Nathan H. “Efficacy and safety of tadalafil in men with erectile dysfunction with and without hypertension”, Am. J. Hypertens. 15 (Part 2): 143A (2002)).

Vardenafil, as shown at FIG. 2B, is a PDE5 inhibitor recently approved for marketing in Europe and US. Vardenafil is characterized by a very high potency in vitro IC50=0.6 nM, compared to sildenafil, 3.0 nM). Pharmacokinetic data for vardenafil were obtained in two randomized, double-blind, placebo-controlled studies with a single oral dose of 10, 20 and 40 mg. The Tmax of vardenafil was 0.7-0.9 h. As with sildenafil, the absorption of vardenafil is delayed if taken after a meal containing >30% fat. Thus, practically, patients should be advised to use vardenafil on an empty stomach to maximize its efficacy (see e.g., Stark S. et al “Vardenafil increased penile rigidity and tumescence in men with erectile dysfunction after single oral dose” Eur. Urol. 40: 181-190 (2001); and Klotz T. et al “Vardenafil increased penile rigidity and tumescence in erectile dysfunction patients: a RigiScan and pharmacokinetic study”, World J. Urol. 19: 32-39 (2001)).

Phase II trials showed that vardenafil was effective in men with severe ED after nerve-sparing radical prostatectomy. After 3 months using 10- or 20-mg doses, patients recorded successful penetration and maintenance of erection significantly more often than placebo-treated men 47% compared with 22%, and 36% compared with 10%, respectively, for each end point (see e.g., “Vardenafil effective and safe as ED therapy in men with prostatectomy, CAD and hypertension” Prous Daily Essentials, 27 Feb. 2002). Data from two phase III studies were pooled to evaluate the safety and efficacy in hypertensive men with mild-to-moderate ED. The drug was dosed at 5, 10 or 20 mg, and all three groups reported results far superior to placebo. Side effects were generally mild, as noted above, and did not occur more frequently in the hypertensive patient population. A smaller study showed that a single 10-mg dose of vardenafil did not increase the risk of exercise-induced cardiac ischemia in patients with stable coronary artery disease.

There are other PDE5 inhibitors in earlier stages of clinical development, and several companies have preclinical discovery programs. FIGS. 2D-F illustrate structures of E-8010, Zaprinast, and E-4021, respectively. Pfizer has reported that a “second-generation” PDE5 inhibitor, UK357903, is now in phase II trials for ED. Tanabe is investigating avanafil in phase II trials for ED and FSD. Dong-A Pharmaceutical entered DA-8159 into phase II clinical trials for ED. DA-8159 is a pyrazolopyrimidinone that has shown erectogenic activity after oral administration of 0.3-1.0 mg kg-1 to rats. In anesthetized dogs, intravenous administration of 1-300 μg kg-1 potentiated an increase in intracavernosal pressure in a dose-related manner. Eisai Pharmaceutical entered E-8010 into phase I clinical trials for ED (Information obtained from the Investigational Drugs Database (IDDB, www.iddb3.com)).

While the invention is described in some detail with specific reference to a single-preferred embodiment and certain alternatives, there is no intent to limit the invention to that particular embodiment or those specific alternatives. For instance, those skilled in the art will appreciate that modifications may be made to the preferred embodiment without departing from the teachings of the present invention.

1. A method for treating schizophrenia in a patient which comprises administration to the patient of an effective amount of a phosphodiesterase type 5 (PDE5) inhibitor, or a pharmaceutically acceptable salt, solvate, or composition thereof for treating schizophrenia.

2. The method of claim 1, wherein the PDE5 inhibitor comprises a selected one of sildenafil, vardenafil, tadalafil, E-8010, zaprinast, and E-4021.

3. The method of claim 1, wherein the PDE5 inhibitor is administered orally.

4. The method of claim 1, wherein the PDE5 inhibitor is administered together with one or more conventional antipsychotic medications.

5. The method of claim 4, wherein said one or more conventional antipsychotic medications include selected ones of risperidone, olanzapine, quetiapine, ziprasidone, aripiprazole, clozapine, haloperidol, and fluphenazine.

6. A method of treating schizophrenia in a human being comprising administering to said human a phosphodiesterase type 5 (PDES) inhibiting compound for treating schizophrenia.

7. The method of claim 6, wherein the PDE5 inhibiting compound comprises a selected one of sildenafil, vardenafil, tadalafil, E-8010, zaprinast, and E-4021.

8. A method for treatment of schizophrenia in a mammal by administering to the mammal a cyclic guanosine 3′,5′-monophosphate phosphodiesterase type five (cGMP PDE V) inhibitor, or a pharmaceutically acceptable salt, solvate, or composition thereof for the treatment of schizophrenia.

9. The method of claim 8, wherein the cGMP PDE V inhibitor comprises a selected one of sildenafil, vardenafil, tadalafil, E-8010, zaprinast, and E-4021.

10. The method of claim 8, wherein the cGMP PDE V inhibitor is administered together with one or more conventional antipsychotic medications.

11. The method of claim 10, wherein said one or more conventional antipsychotic medications include selected ones of risperidone, olanzapine, quetiapine, ziprasidone, aripiprazole, clozapine, haloperidol, and fluphenazine.

12. The method of claim 8, wherein the cGMP PDE V inhibitor is administered orally.

13. The method of claim 8, wherein the cGMP PDE V inhibitor provides therapeutic effects in treatment of cognitive symptoms of schizophrenia.

14. The method of claim 8, wherein the cGMP PDE V inhibitor provides therapeutic effects in treatment of negative symptoms of schizophrenia.

15. The method of claim 1, wherein the PDE5 inhibitor provides therapeutic effects in treatment of cognitive symptoms of schizophrenia.

16. The method of claim 1, wherein the PDE5 inhibitor provides therapeutic effects in treatment of negative symptoms of schizophrenia.

17. The method of claim 6, wherein the PDE5 inhibiting compound provides therapeutic effects in treatment of cognitive symptoms of schizophrenia.

18. The method of claim 6, wherein the PDE5 inhibiting compound provides therapeutic effects in treatment of negative symptoms of schizophrenia.
